(19) United States
(12) Patent Application Publication
(10) Pub. No.: US 2008/0313821 A1
(43) Pub. Date: Dec. 25, 2008

(54) Dyeing processes comprising at least one multi-carbosite, multi-group coupling agent for wash-protecting the color of artificially dyed keratin fibers

(76) Inventors: Boris Lalleman, Paris (FR); Franck Giroud, Chamoux sur Gelon (FR); Gwenaelle Jegou, Saint Michel sur Orge (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/149,297

(22) Filed: Apr. 30, 2008

Related U.S. Application Data
(60) Provisional application No. 60/924,391, filed on May 11, 2007.

(30) Foreign Application Priority Data
Apr. 30, 2007 (FR) 0754798

(51) Int. Cl. A61K 8/30; D06P 5/04; A61Q 5/00

(52) U.S. Cl. 8/408; 8/442; 8/115.51; 8/405

(57) ABSTRACT

Disclosed herein is a cosmetic process for wash-protecting the color of artificially dyed keratin fibers, comprising applying to the keratin fibers at least one composition comprising at least one multi-carbosite, multi-group coupling agent, the coupling agent being an electrophilic hydrocarbon-based compound comprising neither an aldehyde group, nor a carboxylic acid group, nor a formol-generating group, the coupling agent comprising at least two identical reactive groups, the reactive groups not being carried by the same atom, if said atom is a carbon atom, and the coupling agent having a molecular weight of less than 500 g/mol.

This application claims benefit of U.S. Provisional Application No. 60/924,391, filed May 11, 2007, the contents of which are incorporated herein by reference. This application also claims benefit of priority under 35 U.S.C. § 119 to French Patent Application No. 0754798, filed Apr. 30, 2007, the contents of which are also incorporated herein by reference.

Disclosed herein is a process for wash-protecting the color of artificially dyed keratin fibers, for example, human keratin fibers, such as human hair, comprising applying a composition comprising at least one specific multi-carbosite, multi-group coupling agent.

It is known practice to dye keratin fibers, for instance human keratin fibers, such as the hair, with dye compositions comprising oxidation dye precursors, generally known as oxidation bases. These oxidation bases are colorless or weakly colored compounds which, when combined with oxidizing products, give rise to colored compounds via a process of oxidative condensation. It is also known that the shades obtained with these oxidation bases can be varied by combining them with couplers or coloration modifiers. The variety of molecules used as oxidation bases and couplers can allow a wide range of colors to be obtained.

It is also known practice to dye keratin fibers by direct dyeing. The process conventionally used in direct dyeing comprises applying to the keratin fibers direct dyes, which are colored and coloring molecules that have an affinity for the fibers, in leaving them to act, and then in rinsing the fibers.

However, the colorings resulting therefrom, for instance, may be chromatic colorings, but may be, however, temporary or semi-permanent. The nature of the interactions that bind the direct dyes to the keratin fiber and their desorption from the surface and/or the core of the fiber are responsible for their weak dyeing power and their poor wash-fastness.

The artificial color of the hair provided by a direct or oxidation dyeing treatment gradually fades as a result of repeated washing, and leads to fading of the coloration of the hair overtime. Furthermore, the use of commercial rinse-out and leave-in care products does not sufficiently improve the fastness of the artificial color of the hair.

Thus, there is a need in the art for developing methods for protecting the artificial color against the effect of repeated washing, under mild conditions compatible with the dyed hair, for example in terms of temperature.

Short-chain aldehydes having a molecular weight of less than 500 g/mol, for instance formaldehyde, for stabilizing the color of dyed hair is described in German Patent Application No. 100 48 922. However, aldehydes, such as formaldehyde, release stinging and/or suffocating odors that are a great hindrance in a cosmetic product.

The present inventors have discovered, surprisingly, that the use of at least one multi-carbosite, multi-group coupling agent as defined below makes it possible to wash-protect the artificial color of keratin fibers under mild conditions compatible with the dyed hair without releasing stinging and/or suffocating odors.

The various embodiments of the present disclosure are described in more detail below. All meanings and definitions provided herein with respect to the compounds used in the present disclosure are applicable to all embodiments of the present disclosure.

As used herein, “human keratin fibers” is intended to mean head hair, for example beard or moustache hairs, eyelashes or eyebrows.

As used herein, “artificially dyed keratin fibers” is intended to mean keratin fibers dyed by a direct dyeing process or by an oxidation dyeing process.

As used herein, “washing” is intended to mean at least one application on the keratin fibers of an aqueous rinse-out composition, most commonly a detergent composition, such as shampoo. It is also understood to mean bathing, such as in the sea or in a swimming pool.

One aspect of the present disclosure is therefore the use of at least one multi-carbosite, multi-group coupling agent as defined below, as an agent for protecting the color of artificially dyed keratin fibers, such as human keratin fibers, including the hair, with respect to washing.

Another aspect of the present disclosure is a process for wash-protecting the color of artificially dyed keratin fibers, comprising applying to the fibers at least one composition comprising at least one multi-carbosite, multi-group coupling agent as defined below.

Also disclosed herein is a process for wash-protecting the color of artificially dyed keratin fibers, comprising applying to the fibers, before, during or after dyeing, at least one composition comprising, in a cosmetically acceptable medium, at least one multi-carbosite, multi-group coupling agent as defined below.

Furthermore, the protection provided by the treatment according to the present disclosure can be long-lasting and does not necessarily require frequent reapplications of the product.

Yet another aspect of the present disclosure relates to a multistep dyeing process comprising applying to the keratin fibers, for example human keratin fibers, such as the hair, a direct or oxidation dye composition (A) for a period of time sufficient to develop the color, and following or preceding this application with the application of a composition (B) comprising, in a cosmetically acceptable medium, at least one multi-carbosite, multi-group coupling agent as defined below.

Another aspect of the present disclosure relates to a one-step dyeing process comprising applying to the keratin fibers, for instance human keratin fibers, such as the hair, a direct or oxidation dye composition (A′) for a period of time sufficient to develop the color, this composition (A′) comprising, in a cosmetically acceptable medium, at least one multi-carbosite, multi-group coupling agent as defined below.

The at least one multi-carbosite, multi-group coupling agent, as disclosed herein, is an electrophilic hydrocarbon-based compound, with the proviso that it does not comprise an aldehyde group, a carboxylic acid group or salts thereof, a formol-generating group, or an ammonium group, and wherein the coupling agent has a molecular weight of less than 500 g/mol.

As used herein, “hydrocarbon-based compound” is intended to mean a compound comprising carbon and hydrogen atoms and, optionally, at least one atom other than silicon, carbon and hydrogen. The at least one atom can be, for example, chosen from sulfur, phosphorus, halogen, nitrogen and oxygen atoms.

As used herein, “electrophilic compound” or “electrophilic reactive group” is intended to mean any compound or reactive group which lacks negative charges and/or has at least one empty orbital.

The intended meanings of the following terms are:
“multi-group”: comprising at least two identical reactive groups;
“reactive group”: a chemical group capable of reacting so as to form covalent bonds with nucleophilic groups;
“multi-carbosite”: the fact that the reactive groups are not carried by the same atom, if said atom is a carbon atom;
“formol-generating group”: any group capable of releasing formol by at least one of the following processes: increase in temperature of at least 10° C., modification of pH of at least one hydrolysis unit; and
“ammonium group”: any group having a positive charge on the nitrogen.

According to at least one embodiment of the present disclosure, the reactive groups of the at least one multi-carbosite, multi-group coupling agent do not react with the compounds present in the cosmetic compositions so as to form covalent bonds, before the application of the cosmetic composition to keratin materials, such as the hair.

The reactive chemical groups of the multi-carbosite, multi-group coupling agent are capable of reacting so as to form covalent bonds, after the application of the cosmetic composition to keratin materials, for example the hair, with the nucleophilic groups of the dyes present on said keratin materials and, optionally, with the nucleophilic groups of said keratin materials.

The reactive chemical groups present in the dyes are, for example, the groups —X(H)ₙ(R)ₚ₋ₙ₋₁ with X═O, N, S or COO, and p is the valency of X, R is a monovalent radical, and n=1 or 2, such as hydroxyl and amine groups.

The reactive chemical groups present on keratin materials such as the hair are, for instance the groups —X(H)ₙ(R)ₚ₋ₙ₋₁ with X═O, N, S or COO, and p is the valency of X, R is a monovalent radical, n=0, 1 or 2, and p−n−1=0, such as hydroxyl, amine, carboxyl and thiol groups.

The multi-carbosite, multi-group coupling agents, according to the present disclosure, comprise at least one group capable of reacting with the reactive chemical groups of the at least one dye present on the hair.

The reactive groups of the multi-carbosite, multi-group coupling agent can be, for example, chosen from:
epoxides,
optionally hydroxylated (C₂-C₃₀)alkyl sulfates,
aziridines,
activated cyclopropanes, for example 1,1-dicyanocyclopropane,
vinyls and activated vinyls, such as acrylonitrile, acrylic and methacrylic esters, crotonic esters, cinnamic esters, styrene and derivatives, butadiene, vinyl ethers, vinyl ketones, maleic esters, vinyl sulfones, maleimides, cyano (meth)acrylates and (meth)acrylamides,
alkyl esters, alkylamides,
anhydrides,
acyl (or haloformyl) halides, for example acyl chloride,
acetals and hemiacetals,
ketones, alpha-hydroxy ketones, alpha halo ketones,
lactones, thiolactones,
isocyanates,
isothiocyanate,
imides, for instance succinimide, such as N-hydroxysuccinimide esters,
imidates
thiosulfates,
oxazines and oxazolines,
oxaziniums and oxazolinium,
C₆ to C₃₀ alkyl groups, aryl and aralkyl halides of formula RX wherein X is chosen from I, Br and Cl,
halides of an unsaturated ring which is carbon-based, or heterocycles, for example chlorotriazines, chloropyrimidines, chloroquinoxalines, chlorobenzotriazoles, chlorohydrins,
sulfonyl halides (—SO₂X), wherein X is, for example, Cl or F,
(C₂-C₃₀)alkyl phosphonium,
phenols, such as in the form of ortho-diphenol, quinone,
(C₁-C₃₀)alkyl nitrophenyl carbonates,
(C₁-C₃₀)alkyl or phenyl tosylates,
alkyl chloroformates,
enol esters,
sulfonamides, and
ring opening reactants, for instance the carbonate or cyclic carbonate as described in French Patent No. 2 846 879, N-carboxyanhydrides.

The reactive groups are also described in a survey and critique of the literature on crosslinking agents, Bioconjugate Techniques by Greg T. Hermanson, published in 1996 by Academic Press and Mechanisms, and as related to wool keratin, Everett H. Hinton, Jr., Textile Research Journal, April 1974, p 233-292.

According to at least one embodiment, the reactive groups of the at least one multi-carbosite, multi-group coupling agent can be chosen from the following groups: aziridine, epoxides, (C₂-C₃₀)alkyl sulfate, vinyl, activated vinyl, ketones, for example alpha-hydroxy ketones and alpha-halo ketones, lactones, oxalate, thiolactones, isocyanates, isothiocyanate, oxazine, oxazoline, halide of an unsaturated ring which is carbon-based, or a heterocycle, sulfonyl halide, and (C₂-C₃₀)alkyl phosphonium.

According to another embodiment, the reactive groups can be chosen from cyanoacrylate, ketone, alpha-hydroxy ketones, chlorotriazine, triethanolamine trisulphate and lactone.

The multi-carbosite, multi-group coupling agent of the present disclosure, in at least one embodiment, exhibits at least one condition below:
does not induce an extension of the electron conjugation on the dye (or of the product for coupling the dye molecules),
is water-soluble or liposoluble, and in at least one embodiment, is water-soluble,
is stable under aqueous conditions at ambient temperature,
has a molecular weight of less than 500 g/mol, for example, less than 250 g/mol, such as less than 150 g/mol.

The water-soluble multi-carbosite, multi-group coupling agents, as disclosed herein, are soluble in water at a concentration of greater than or equal to 0.1% by weight in water at 25° C., i.e., they form, under these conditions, an isotropic solution that is transparent to the eye.

The at least one multi-carbosite, multi-group coupling agent, in at least one embodiment, is chosen from 1,3-butadiene diepoxide, triethanolamine trisulfate, ethylenediaminetetraacetic dianhydride, dimethyl oxalate and sodium sulfosuccinimidyl-4-(N-maleimidomethyl)cyclohexane-1-carboxylate.

The at least one multi-carbosite, multi-group coupling agent, as disclosed herein, may be present in an amount ranging from 0.01% to 20% by weight, for example from 0.1% to 10% by weight, relative to the total weight of the composition.

In at least one embodiment, the composition comprises a cosmetically acceptable medium.

The cosmetically acceptable medium of the compositions for wash-protecting the color of keratin fibers, according to the present disclosure, may comprise, for example, water or of a mixture of water and at least one cosmetically acceptable organic solvent. Non-limiting examples of organic solvent may include C₁-C₄ lower alkanols, such as ethanol and isopropanol; polyols and polyol ethers, such as 2-butoxyethanol, propylene glycol, propylene glycol monomethyl ether, diethylene glycol monoethyl ether and diethylene glycol monomethyl ether, and mixtures thereof.

The at least one solvent can be present in an amount ranging from 1% to 40% by weight, such as from 3% to 10% by weight, relative to the total weight of the composition.

The compositions disclosed herein may also comprise at least one adjuvant conventionally used in hair treatment compositions. Useful adjuvants may include: anionic, cationic, nonionic, amphoteric, zwitterionic surfactants and mixtures thereof; anionic, cationic, nonionic, amphoteric, zwitterionic polymers and mixtures thereof; inorganic and organic thickeners, such as anionic, cationic, nonionic and amphoteric polymeric associative thickeners; penetrating agents; sequestering agents; fragrances; buffers; dispersants; conditioning agents, for example modified and unmodified, volatile and nonvolatile silicones; film-forming agents; ceramides; preservatives; and opacifiers.

According to at least one embodiment of the present disclosure, the compositions may further comprise at least one agent for protecting keratin fibers against the effects of atmospheric agents, such as light.

The at least one agent for protecting keratin fibers may be any active agent that can be used for preventing or limiting degradation of keratin fibers, for example the hair, caused by atmospheric attacks, such as light.

Thus, the at least one agent for protecting keratin fibers may be chosen from organic UV screening agents, free-radical scavengers and antioxidants.

As used herein, the term “free-radical scavenger” is intended to mean any compound capable of trapping free radicals.

The organic UV screening agents (systems for screening out UV radiation) are, in at least one embodiment, chosen from water-soluble and liposoluble, silicone and nonsilicone screening agents.

Non-limiting examples of organic UV screening agents include: dibenzoylmethane derivatives; anthranilates; cinnamic derivatives; salicylic derivatives; camphor derivatives; benzophenone derivatives; β,β-diphenylacrylate derivatives; triazine derivatives; benzotriazole derivatives; benzalmalonate derivatives; benzimidazole derivatives; imidazolines; bis-benzoazolyl derivatives as described in European Patent No. 0 669 323 and U.S. Pat. No. 2,463,264; p-aminobenzoic acid (PABA) derivatives; benzoxazole derivatives as described in European Patent Application Nos. 0 832 642, 1 027 883, and 1 300 137, and German Application No. 10 162 844; screening polymers and screening silicones such as those described in International Application Publication No. WO 93/04665; dimers derived from α-alkylstryene, such as those described in German Patent Application No. 19 855 649; 4,4-diarylbutadienes such as those described in European Patent Application Nos. 0 967 200, 1 008 586, 1 133 980 and 0 133 981, and German Patent Application Nos. 19 746 654, and 19 755 649, and mixtures thereof.

Other examples also useful as organic UV screening agents, include, but are not limited to those denoted hereinbelow under their INCI name:

### Para-Aminobenzoic Acid Derivatives:

### PABA,

### Ethyl PABA,

Ethyl dihydroxypropyl PABA,
Ethylhexyl dimethyl PABA sold, for instance under the name “ESCALOL 507” by ISP,

### Glyceryl PABA,

PEG-25 PABA sold, for instance under the name “UVINUL P25” by BASF.

### Cinnamic Derivatives:

Ethylhexyl methoxycinnamate sold, for example under the trade name “PARSOL MCX” by Hoffmann LaRoche,
Isopropyl methoxycinnamate,
Isoamyl methoxycinnamate sold, for instance under the trade name “NEO HELIOPAN E 1000” by Haarmann and Reimer,

### Cinoxate,

DEA methoxycinnamate,
Diisopropyl methylcinnamate,
Glyceryl ethylhexanoate dimethoxycinnamate.

### Dibenzoylmethane Derivatives:

Butylmethoxydibenzoylmethane sold, for example under the trade name “PARSOL 1789” by Hoffmann LaRoche,
Isopropyldibenzoylmethane sold, for instance under the trade name “EUSOLEX 8020” by Merck.

### β,β-Diphenylacrylate Derivatives:

Octocrylene sold, for example under the trade name “UVINUL N539” by BASF,
Etocrylene sold, for instance under the trade name “UVINUL N35” by BASF.

### Benzophenone Derivatives:

Benzophenone-1 sold, for example under the trade name “UVINUL 400” by BASF,
Benzophenone-2 sold, for instance under the trade name “UVINUL D50” by BASF,
Benzophenone-3 or Oxybenzone sold, for example under the trade name “UVINUL M40” by BASF,
Benzophenone-4 sold, for instance under the trade name “UVINUL MS40” by BASF,

### Benzophenone-5,

Benzophenone-6 sold, for example under the trade name “HELISORB 11” by Norquay,
Benzophenone-8 sold, for instance under the trade name “SPECTRA-SORB UV-24” by American Cyanamid,
Benzophenone-9 sold, for example under the trade name “UVINUL DS-49” by BASF,

### Benzophenone-12

n-hexyl 2-(4-diethylamino-2-hydroxybenzoyl)benzoate.

### Benzylidenecamphor Derivatives:

3-Benzylidenecamphor manufactured, for example under the name “MEXORYL SD” by Chimex,
4-Methylbenzylidenecamphor sold, for instance under the name “EUSOLEX 6300” by Merck,
Benzylidenecamphorsulfonic acid manufactured, for example under the name “MEXORYL SL” by Chimex,
Camphor benzalkonium methosulfate manufactured, for instance under the name “MEXORYL SO” by Chimex,
Terephthalylidenedicamphorsulfonic acid manufactured, for example under the name “MEXORYL SX” by Chimex,
Polyacrylamidomethylbenzylidenecamphor manufactured, for instance under the name “MEXORYL SW” by Chimex.

### Phenylbenzimidazole Derivatives:

Phenylbenzimidazolesulfonic acid sold, for example under the trade name “EUSOLEX 232” by Merck,
Disodium phenyl dibenzimidazole tetrasulfonate sold, for instance under the trade name “NEO HELIOPAN AP” by Haarmann and Reimer.

### Phenylbenzotriazole Derivatives:

Drometrizole trisiloxane sold, for example under the name “SILATRIZOLE” by Rhodia Chimie,
Methylenebis(benzotriazolyl)tetramethylbutylphenol sold, for instance in solid form under the trade name “MIXXIM BB/100” by Fairmount Chemical, or in micronized form as an aqueous dispersion under the trade name “TINOSORB M” by Ciba Specialty Chemicals.

### Triazine Derivatives:

Bisethylhexyloxyphenol Methoxyphenyl Triazine sold, for example under the trade name “TINOSORB S” by Ciba Geigy, Ethylhexyltriazone sold, for instance under the trade name “UVINUL T150” by BASF,
Diethylhexylbutamidotriazone sold, for example under the trade name “UVASORB HEB” by Sigma 3V,
2,4,6-tris(diisobutyl 4′-aminobenzalmalonate)-s-triazine.

### Imidazoline Derivatives:

Ethylhexyldimethoxybenzylidenedioxoimidazoline propionate.

### Benzalmalonate Derivatives:

Polyorganosiloxane containing benzalmalonate functions, for instance Polysilicone-15, sold under the trade name “PARSOL SLX” by Hoffmann LaRoche

### 4,4-Diarylbutadiene Derivatives:

1,1-Dicarboxy(2,2′-dimethylpropyl)-4,4-diphenylbutadiene

### Benzoxazole Derivatives:

2,4-bis[5-(1-dimethylpropyl)benzoxazol-2-yl(4-phenyl)imino]-6-(2-ethylhexyl)imino-1,3,5-triazine sold, for example under the name UVASORB K2A by Sigma 3V
and mixtures thereof.

Liposoluble (or lipophilic) organic UV screening agents that are suitable for use, according to the present disclosure, include but are not limited to:
ethylhexyl methoxycinnamate,
butylmethoxydibenzoylmethane,
octocrylene,
benzophenone-3,
n-hexyl 2-(4-diethylamino-2-hydroxybenzoyl)benzoate,
4-methylbenzylidenecamphor,
ethylhexyl triazone,
bisethylhexyloxyphenol methoxyphenyl triazine,
diethylhexyl butamido triazone,
drometrizole trisiloxane,
polysilicone-15,
1,1-dicarboxy(2,2′-dimethylpropyl)-4,4-diphenylbutadiene, and
2,4-bis[5-1 (dimethylpropyl)benzoxazol-2-yl(4-phenyl)imino]-6-(2-ethylhexyl)imino-1,3,5-triazine.

Water-soluble (or hydrophilic) organic UV screening agents that may be useful according to the present disclosure include:
PABA,
PEG-25 PABA
benzylidenecamphorsulphonic acid,
camphorbenzalkonium methosulphate,
terephthalylidenedicamphorsulphonic acid,
phenylbenzimidazolesulphonic acid,
disodium phenyldibenzimidazoletetrasulphonate,
benzophenone-4, and
benzophenone-5.

The free-radical scavengers that may be used in the composition according to the present disclosure can be chosen from, in addition to certain antipollultion agents previously mentioned, vitamin E and its derivatives, such as tocopheryl acetate; bioflavonoids; coenzyme Q10 or ubiqinone; certain enzymes, such as catalase, superoxide dismutase and wheatgerm extracts comprising same, lactoperoxidase, glutathione peroxidase and quinone reductases; glutathione; benzylidenecamphor; benzylcyclanones; substituted naphthalenones; pidolates; phytantriol; gamma-oryzanol; guanosine; lignans; and melatonin.

Useful antioxidants include, but are not limited to phenols, such as BHA (tert-butyl-4-hydroxyanisole), BHT (2,6-di-tert-butyl-p-cresol), TBHQ (tert-butylhydroquinone), polyphenols such as proanthocyanidol oligomers and flavonoids, hindered amines known under the generic term HALS (Hindered Amine Light Stabilizer) such as tetraminopiperidine, erythorbic acid, polyamines such as spermine, cysteine, glutathione, superoxide dismutase and lactoferrin.

In at least one embodiment of the present disclosure, the at least one agent for protecting keratin fibers from atmospheric attacks, such as the light are chosen from organic UV screening agents.

According to the present disclosure, the at least one agent for protecting keratin fibers from atmospheric attacks, such as the light, may be present in an amount ranging from 0.2% to 20% by weight, relative to the total weight of the composition.

The compositions according to the present disclosure, may also comprise at least one conditioning agent.

As disclosed herein, “conditioning agent” is intended to mean any agent whose function is to improve the cosmetic properties of the hair, such as the softness, disentangling, feel, smoothness and static electricity.

The at least one conditioning agent may be in liquid, semi-solid or solid form, such as, oils, waxes or gums.

Useful non-limiting examples of the at least one conditioning agent may include synthetic oils such as polyolefins, plant oils, fluoro and perfluoro oils, natural and synthetic waxes, silicones, nonpolysaccharide cationic polymers, compounds of ceramide type, cationic surfactants, fatty amines, saturated fatty acids and fatty acid esters other than those of the disclosure, and mixtures thereof.

The synthetic oils that may be used in the present disclosure may, in at least one embodiment, be chosen from polyolefins, for example poly-α-olefins, and further for example hydrogenated or nonhydrogenated polybutene type, such as hydrogenated or nonhydrogenated polyisobutene.

In at least one embodiment, isobutylene oligomers of molecular weight less than 1,000 and mixtures thereof with polyisobutylenes of molecular weight greater than 1,000, for example ranging from 1,000 to 15,000, may be used.

Examples of poly-α-olefins include, but are not limited to those sold, for example, under the name PERMETHYL 99 A, 101 A, 102 A, 104 A (n=16) and 106 A (n=38) by the company Presperse Inc, or alternatively, the products sold, for instance under the name ARLAMOL HD (n=3) by the company ICI (n denoting the degree of polymerization), of hydrogenated or nonhydrogenated polydecene type.

Such products are sold, for example, under the names ETHYLFLO by the company Ethyl Corp., and ARLAMOL PAO by the company ICI.

The animal or plant oils, which may be used in at least one embodiment of the present disclosure, may be chosen from, by way of non-limiting example, sunflower oil, maize oil, soya oil, avocado oil, jojoba oil, marrow oil, grapeseed oil, sesame oil, hazelnut oil, fish oils, glyceryl tricaprocaprylate, and plant or animal oils of formula R₉COOR₁₀ wherein R₉ is a saturated higher fatty acid residue-comprising from 7 to 29 carbon atoms and R₁₀ is a linear or branched hydrocarbon-based chain comprising from 3 to 30 carbon atoms, for example alkyl or alkenyl, and further for example, purcellin oil.

Natural or synthetic essential oils, such as, eucalyptus oil, lavandine oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil may also be used.

The waxes can be natural (animal or plant) or synthetic substances that are solid at ambient-temperature (20°-25° C.). They are insoluble in water, soluble in oils and are capable of forming a water-repellent film.

For the definition of waxes, reference may be made to the document, P. D. Dorgan, Drug and Cosmetic Industry, December 1983, pp. 30-33.

Non-limiting examples of waxes, which may be used according to the present disclosure include those chosen from: carnauba wax, candelilla wax, alfa wax, paraffin wax, ozokerite, plant waxes, such as olive tree wax, rice wax, hydrogenated jojoba wax and absolute waxes of flowers, such as essential oil of blackcurrant flower sold by the company Bertin (France), animal waxes, such as beeswaxes, and modified beeswaxes (cerabellina); other waxes or waxy starting materials which may be used are, for example, those sold by the company Sophim under the reference M82, and polyethylene waxes or polyolefin waxes in general.

According to at least one embodiment, the at least one conditioning agent is chosen from cationic polymers and silicones.

Useful cationic polymers, in accordance with the present disclosure, may be chosen from all those already known to improve the cosmetic properties of hair treated with detergent compositions, for example, those described in European Patent Application No. 0 337 354 and in French Patent Application Nos. 2 270 846, 2 383 660, 2 598 611, 2 470 596 and 2 519 863.

As used herein, “nonsaccharide polymers” is understood to mean polymers that do not comprise a glycoside bond between monosaccharides.

As used herein, “cationic polymer” is any polymer comprising cationic groups and/or groups that may be ionized into cationic groups.

Among the cationic polymers that may be used in the present disclosure, non-limiting mention may be made of those which contain primary, secondary, tertiary and/or quaternary amines capable of forming part of the main polymer chain or of being borne by a side substituent directly attached thereto.

In at least one embodiment, the cationic polymers that may be used herein, have a number-average molecular mass ranging from 500 to 5×10⁶, for example, ranging from 10³ to 3×10⁶.

Suitable cationic polymers include, by way of non-limiting example, polymers of the polyamine, polyamino amide and polyquaternary ammonium type.

The polymers of the polyamine, polyamido amide and polyquaternary ammonium type that can be used herein, may be chosen, for example, from those described in French Patent Nos. 2 505 348 and 2 542 997. Among these polymers, the following may be cited in a non-limiting manner:

(1) homopolymers or copolymers derived from acrylic or methacrylic esters or amides and comprising at least one unit of the following formulae:

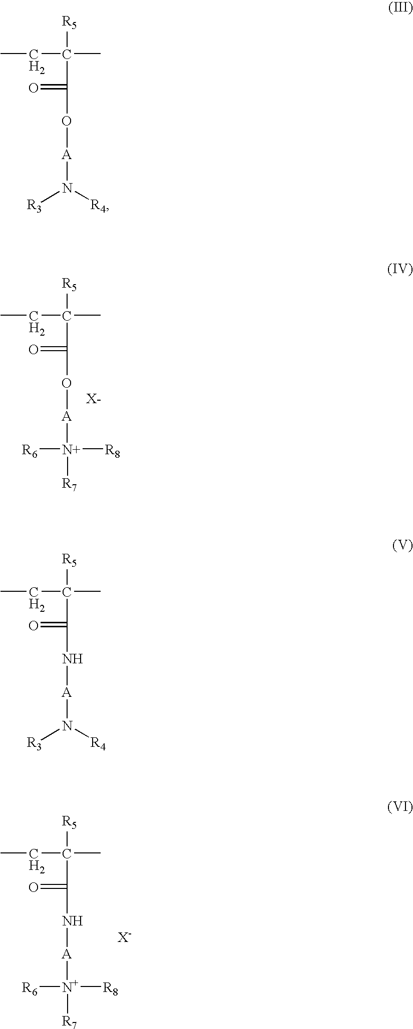

wherein:
R₃ and R₄, which may be identical or different, are chosen from hydrogen and C₁-C₆ alkyl groups, for example methyl or ethyl;
R₅, which may be identical or different, is chosen from hydrogen and CH₃ radicals;
A, which may be identical or different, is chosen form linear and branched C₁-C₆ alkyl groups, for example ethyl or propyl, and hydroxyalkyl groups comprising from 1 to 4 carbon atoms;
R₆, R₇ and R₈, which may be identical or different, are chosen from C₁-C₁₈ alkyl groups and benzyl radicals, for example a C₁-C₆ alkyl group;
X is chosen from anions derived from a mineral or organic acid, such as a methosulfate anion and halides such as chloride or bromide.

The copolymers of family (1) may also contain at least one unit derived from comonomers which may be chosen from acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters. Examples of copolymers of family (1) that are suitable for use in accordance with the present disclosure include, but are not limited to:

copolymers of acrylamide and of dimethylaminoethyl methacrylate quaternized with dimethyl sulfate or with a dimethyl halide, such as the product sold under the name HERCOFLOC by the company Hercules,

the copolymers of acrylamide and of methacryloyloxyethyltrimethylammonium chloride described, for example, in European Patent Application No. 0 080 976 and sold, for instance, under the name BINA QUAT P 100 by the company Ciba Geigy,

the copolymer of acrylamide and of methacryloyloxyethyltrimethylammonium methosulfate sold, for example, under the name RETEN by the company Hercules,

quaternized or nonquaternized vinylpyrrolidone/dialkylaminoalkyl acrylate or methacrylate copolymers, such as the products sold, for instance, under the name GAFQUAT by the company ISP, such as, GAFQUAT 734 or GAFQUAT 755, or alternatively the products COPOLYMER 845, 958 and 937. These polymers are described in detail in French Patent Nos. 2 077 143 and 2 393 573,

dimethylaminoethyl methacrylate/vinylcaprolactam/vinylpyrrolidone terpolymers, such as the product sold, for example, under the name GAFFIX VC 713 by the company ISP,

vinylpyrrolidone/methacrylamidopropyldimethylamine copolymers sold, for instance, under the name STYLEZE CC 10 by ISP, and

quaternized vinylpyrrolidone/dimethylaminopropylmethacrylamide copolymers, such as the product sold, for example, under the name GAFQUAT HS 100 by the company ISP.

(2) Polymers comprising piperazinyl units and divalent alkylene or hydroxyalkylene radicals comprising straight or branched chains, which may be optionally interrupted by at least one atom chosen from oxygen, sulfur and nitrogen atoms, or by aromatic and heterocyclic rings, as well as the oxidation and/or quaternization products of these polymers. Such polymers are described, for example, in French Patent Nos. 2 162 025 and 2 280 361.
(3) Water-soluble polyamino amides prepared, for instance, by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in an amount ranging from 0.025 to 0.35 mol per amine group of the polyamino amide; these polyamino amides can be alkylated or, if they comprise at least one tertiary amine function, they can be quaternized. Such polymers are described, for instance, in French Patent Nos. 2 252 840 and 2 368 508.
(4) The polyamino amide derivatives resulting from the condensation of polyalkylene polyamines with polycarboxylic acids followed by alkylation with difunctional agents, for example adipic acid/dialkylaminohydroxyalkyldialkylenetriamine polymers wherein the alkyl radical contains from 1 to 4 carbon atom, for example methyl, ethyl or propyl. These polymers are described, for instance, in French Patent No. 1 583 363.

Among these derivatives, non-limiting mention may be made, for example of the adipic acid/dimethylaminohydroxypropyl/diethylenetriamine polymers sold under the name CARTARETINE F, F4 or F8 by the company Sandoz.

(5) The polymers obtained by reaction of a polyalkylene polyamine comprising two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid ranges from 0.8:1 to 1.4:1; the polyamino amide that results from reaction with epichlorohydrin in a molar ratio of epichlorohydrin relative to the secondary amine group of the polyamino amide ranges from 0.5:1 to 1.8:1. These polymers are described, for example, in U.S. Pat. Nos. 3,227,615 and 2 961 347.

Polymers of this type are sold, for instance, under the name HERCOSETT 57 by the company Hercules Inc. or alternatively under the name PD 170 or DELSETTE 101 by the company Hercules in the case of the adipic acid/epoxypropyl/diethylenetriamine copolymer.

(6) Cyclopolymers of alkyldiallylamine or of dialkyldiallylammonium, such as the homopolymers or copolymers comprising, as main constituent of the chain, units chosen from the formulae (VII) and/or (VIII):

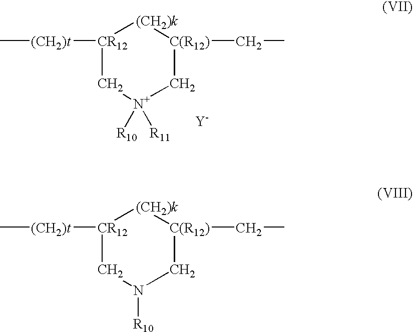

wherein:

k and t are 0 or 1;

the sum k+t is 1;

R₁₂ is chosen from a hydrogen atom and methyl radicals;

R₁₀ and R₁₁, are independently chosen from C₁-C₆ alkyl groups, hydroxy(C₁-C₅)alkyl groups, lower (C₁-C₄) amidoalkyl groups, or R₁₀ and R₁₁ may form together with the nitrogen atom to which they are attached, heterocyclic groups such as piperidyl or morpholinyl;

Y⁻ is chosen from anions such as bromide, chloride, acetate, borate, citrate, tartrate, bisulfate, bisulfite, sulfate and phosphate. These polymers are described, for example in French Patent No. 2 080 759 and in its Certificate of Addition 2 190 406.

According to at least one embodiment of the present disclosure, R₁₀ and R₁₁, are independently chosen from C₁-C₄ alkyl groups.

Among the polymers defined above, non-limiting mention may be made, for example, of the dimethyldiallylammonium chloride homopolymer sold, for instance, under the name MERQUAT 100 by the company Nalco (and its homologues of low weight-average molecular mass) and the copolymers of diallyldimethylammonium chloride and of acrylamide, sold, for instance under the name MERQUAT 550.

(7) The quaternary diammonium polymer comprising repeating units chosen from those of formula (IX):

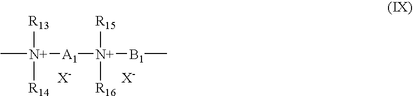

wherein:
R₁₃, R₁₄, R₁₅ and R₁₆, which may be identical or different, are chosen from aliphatic, alicyclic and arylaliphatic radicals comprising from 1 to 20 carbon atoms and lower hydroxyalkylaliphatic radicals, or alternatively R₁₃, R₁₄, R₁₅ and R₁₆, together or separately, form with the nitrogen atoms to which they are attached, heterocycles optionally comprising a second heteroatom other than nitrogen, or alternatively R₁₃, R₁₄, R₁₅ and R₁₆ are chosen from linear and branched C₁-C₆ alkyl radicals substituted with a nitrile, ester, acyl or amide group or a group —CO—O—R₁₇-D or —CO—NH—R₁₇-D wherein R₁₇ is an alkylene and D is a quaternary ammonium group;
A₁ and B₁ are chosen from polymethylene groups comprising from 2 to 20 carbon atoms which may be linear or branched, saturated or unsaturated, and which may comprise, linked to or intercalated in the main chain, at least one aromatic ring or at least one oxygen or sulfur atom or sulfoxide, sulfone, disulfide, amino, alkylamino, hydroxyl, quaternary ammonium, ureido, amide or ester group, and
X⁻ is an anion derived from a mineral or organic acid;
A₁, R₁₃ and R₁₅ can form, with the two nitrogen atoms to which they are attached, a piperazine ring; in addition, if A₁ is a linear or branched, saturated or unsaturated alkylene or hydroxyalkylene radical, B₁ can also be a group (CH₂)ₙₚ—CO-D-OC—(CH₂)ₚ—, wherein p is an integer ranging from about 2 to 20,

wherein D is chosen from:

a) a glycol residue of formula: —O-Z-O—, wherein Z is chosen from linear and branched hydrocarbon-based radicals or a group corresponding to one of the following formulae:

—(CH₂—CH₂—O)ₓ—CH₂—CH₂—

—[CH₂—CH(CH₃)—O]_{y}—CH₂—CH(CH₃)—

wherein x and y are integers ranging from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH—, wherein Y is chosen from linear and branched hydrocarbon-based radicals, or alternatively the divalent radical —CH₂—CH₂—S—S—CH₂—CH₂—;

d) a ureylene group of formula: —NH—CO—NH—.

In at least one embodiment, X⁻ is an anion such as chloride or bromide.

These polymers which may be used in the context of the present disclosure, may have a number-average molecular mass ranging from 1,000 to 100,000.

Such polymers are further described, for example in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

In at least one embodiment, polymers that consist of repeating units corresponding to the formula (a), may be used:

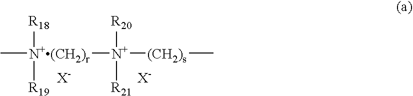

wherein

R₁₈, R₁₉, R₂₀ and R₂₁, which may be identical or different, are chosen from C₁-C₄ alkyl groups and hydroxy(C₁-C₄)alkyl radicals;

r and s are integers ranging from 2 to 20; and

X⁻ is an anion derived from a mineral or organic acid.

In another embodiment, a compound of formula (a) wherein R₁₈, R₁₉, R₂₀ and R₂, are methyl radicals and r is 3, s is 6 and X is Cl, referred to as hexadimethrine chloride according to the INCI nomenclature (CTFA), may be used.

(8) Polyquaternary ammonium polymers comprising units of formula (X):

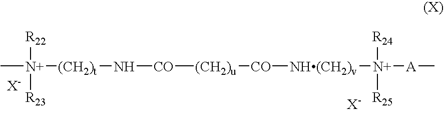

wherein:
R₂₂, R₂₃, R₂₄ and R₂₅, which may be identical or different, are chosen from a hydrogen atom and methyl, ethyl, propyl, β-hydroxyethyl, β-hydroxypropyl and —CH₂CH₂(OCH₂CH₂)ₚOH radicals,

wherein p is 0 or an integer ranging from 1 to 6, with the proviso that R₂₂, R₂₃, R₂₄ and R₂₅ are not simultaneously hydrogen,

t and u, which may be identical or different, are integers ranging from 1 to 6,

v is 0 or an integer ranging from 1 to 34,

X⁻ is an anion, such as a halide,

A is chosen from dihalide radicals and in at least one embodiment, is —CH₂—CH₂—O—CH₂—CH₂—.

These compounds are described, for example in European Patent Application No. 0 122 324.

Among these products, non-limiting mention may be made, for example, of MIRAPOL® A 15, MIRAPOL®AD1, MIRAPOL® AZ1 and MIRAPOL® 175 sold by the company Miranol.

(9) Quaternary polymers of vinylpyrrolidone and of vinylimidazole, such as the products sold under the names LUVIQUAT® FC 905, FC 550 and FC 370 by the company BASF.
(10) Crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, for example methylenebisacrylamide. In at least one embodiment, the polymer may be a crosslinked acrylamide/methacryloyloxyethyltrimethylammonium chloride copolymer (20/80 by weight) in the form of a dispersion containing 50% by weight of said copolymer in mineral oil. This dispersion is sold, for example, under the name SALCARE® SC 92 by the company Ciba. In another embodiment, the polymer may be a crosslinked methacryloyloxyethyltrimethylammonium chloride homopolymer containing about 50% by weight of the homopolymer in mineral oil or in a liquid ester. This dispersion is sold, for example under the names SALCARE® SC 95 and SALCARE® SC 96 by the company Ciba.

Other useful cationic polymers that may be used in the context of the present disclosure are cationic proteins and cationic protein hydrolysates, polyalkyleneimines, for example polyethyleneimines, polymers containing vinylpyridine or vinylpyridinium units, condensates of polyamines and of epichlorohydrin, quaternary polyureylenes and chitin derivatives.

In at least one embodiment, cationic cyclopolymers, such as the dimethyldiallylammonium chloride homopolymers or copolymers sold, for instance, under the names MERQUAT 100, MERQUAT 550 and MERQUAT S by the company Nalco, quaternary vinylpyrrolidone and vinylimidazole polymers, and mixtures thereof, may be used.

Suitable silicones that can be used in at least one embodiment of the present disclosure, are polyorganosiloxanes that are insoluble in the composition and that may be in the form of oils, waxes, resins or gums.

The organopolysiloxanes are defined, for example, in greater detail in Walter Noll “Chemistry and Technology of Silicones” (1968) Academic Press. They can be volatile or non-volatile.

Non-limiting examples of volatile silicones that may be used in accordance with the present disclosure include those that have a boiling point ranging from 60° C. to 260° C., for example:

(i) cyclic silicones comprising from 3 to 7 silicon atoms, for example 4 to 5 silicon atoms. These cyclic silicones are, for example, octamethylcyclotetrasiloxane sold under the name VOLATILE SILICONE 7207 by Union Carbide or SILBIONE 70045 V 2 by Rhodia Chimie, decamethylcyclopentasiloxane sold, for example, under the name VOLATILE SILICONE 7158 by Union Carbide, and SILBIONE 70045 V 5 by Rhodia Chimie, and mixtures thereof.

Non-limiting mention may also be made of cyclocopolymers of the dimethylsiloxanes/methylalkylsiloxane type, such as VOLATILE SILICONE FZ 3109 sold, for instance, by the company Union Carbide, having the chemical structure:

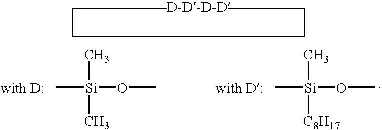

Non-limiting mention may also be made of mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones comprising 2 to 9 silicon atoms and having a viscosity of less than or equal to 5×10⁻⁶ m²/s at 25° C., for example decamethyltetrasiloxane sold under the name SH 200 by the company Toray Silicone. These silicones are also described in the article published in Cosmetics and Toiletries, Vol. 91, Jan. 76, pp. 27-32, Todd & Byers “Volatile Silicone Fluids for Cosmetics.”

In at least one embodiment, non-limiting examples of non-volatile silicones that may be used in the present disclosure include polyalkylsiloxanes, polyarylsiloxanes, polyalkylarylsiloxanes, silicone gums and resins, polyorganosiloxanes modified with organofunctional groups, and mixtures thereof.

Non-limiting examples of non-volatile silicones include, but are not limited to polyalkylsiloxanes, for example, polydimethylsiloxanes comprising trimethylsilyl end groups with a viscosity ranging from 5×10⁻⁶ to 2.5 m²/s at 25° C., for example, ranging from 1×0-5 to 1 m²/s. The viscosity of the silicones is measured, for example, at 25° C. according to ASTM standard 445 Appendix C.

Among these polyalkylsiloxanes, the following commercial products may be cited in a non-limiting manner:

the SILBIONE oils of the 47 and 70 047 series or the MIRASIL oils sold by Rhodia Chimie, such as the oil 70 047 V 500 000;

the oils of the MIRASIL series sold by the company Rhodia Chimie;

the oils of the 200 series from the company Dow Corning, such as DC200 with a viscosity of 60 000 cSt;

the VISCASIL oils from General Electric and certain oils of the SF series (SF 96, SF 18) from General Electric.

Also useful herein are polydimethylsiloxanes comprising dimethylsilanol end groups (Dimethiconol according to the CTFA name) such as the oils of the 48 series from the company Rhodia Chimie.

Non-limiting mention may also be made among this class of polyalkylsiloxanes, for example, products sold under the name ABIL WAX 9800 and 9801 by the company Goldschmidt, which are poly(C₁-C₂₀)alkylsiloxanes.

The polyalkylarylsiloxanes that may be used in the present disclosure may, in at least one embodiment, be chosen from linear and branched polydimethylmethylphenylsiloxanes and polydimethyldiphenylsiloxanes with a viscosity ranging from 1×10⁻⁵ to 5×10⁻² m²/s at 25° C.

Examples of such polyalkylarylsiloxanes include, but are not limited to the products sold under the following names:

the SILBIONE oils of the 70 641 series from Rhodia Chimie;

the oils of the RHODORSIL 70 633 and 763 series from Rhodia Chimie;

the oil Dow Corning 556 Cosmetic Grade Fluid from Dow Corning;

the silicones of the PK series from Bayer, such as the product PK20;

the silicones of the PN and PH series from Bayer, such as the products PN1000 and PH1000;

certain oils of the SF series from General Electric, such as SF 1023, SF 1154, SF 1250 and SF 1265.

Useful silicone gums according to the present disclosure include, but are not limited to polydiorganosiloxanes with high number-average molecular masses ranging from 200,000 to 1,000,000, used alone or as a mixture in a solvent. Non-limiting examples of this solvent include volatile silicones, polydimethylsiloxane (PDMS) oils, polyphenylmethylsiloxane (PPMS) oils, isoparaffins, polyisobutylenes, methylene chloride, pentane, dodecane, tridecane, and mixtures thereof.

Polydiorganosiloxanes which may be used in at least one embodiment of the present disclosure include, but are not limited to:

polydimethylsiloxane,

polydimethylsiloxane/methylvinylsiloxane gums,

polydimethylsiloxane/diphenylsiloxane,

polydimethylsiloxane/phenylmethylsiloxane, and

polydimethylsiloxane/diphenylsiloxane/methylvinylsiloxane.

Products useful in at least one embodiment of the present disclosure are mixtures such as:

mixtures formed from a polydimethylsiloxane hydroxylated at the end of the chain (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;

mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500,000, dissolved in the oil SF 1202 Silicone Fluid corresponding to decamethylcyclopentasiloxane;

mixtures of two PDMSs of different viscosities, for example a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. This product may, for example, comprise 15% of SE 30 gum and 85% of an SF 96 oil.

Useful organopolysiloxane resins, according to the present disclosure include, but are not limited to crosslinked siloxane systems comprising the following moieties:

R₂SiO_{2/2}, R₃SiO_{1/2}, RSiO_{3/2} and SiO_{4/2} wherein R is chosen from C₁-C₁₆ hydrocarbon-based groups and phenyl groups. Among these products are those wherein R is a C₁-C₄ lower alkyl radical, for example methyl, or a phenyl radical.

Non-limiting mention may be made among these resins, for example, of the product sold under the name DOW CORNING 593 or those sold under the names SILICONE FLUID SS 4230 and SS 4267 by the company General Electric, which are silicones of dimethyl/trimethyl siloxane structure.

Other useful resins include, but are not limited to, the trimethyl siloxysilicate type resins sold, for example under the names X22-4914, X21-5034 and X21-5037 by the company Shin-Etsu.

The organomodified silicones that can be used in accordance with the present disclosure may be chosen from silicones as defined above and comprising in their structure at least one organofunctional group attached via a hydrocarbon-based radical.

Examples of organomodified silicones include, but are not limited to polyorganosiloxanes comprising:

polyethyleneoxy and/or polypropyleneoxy groups optionally comprising C₆-C₂₄ alkyl groups, such as the products known as dimethicone copolyol sold by the company Dow Corning under the name DC 1248 or the oils SILWET L 722, L 7500, L 77 and L 711 by the company Union Carbide, and the (C₁₋₂)alkylmethicone copolyol sold by the company Dow Corning under the name Q2 5200;

substituted or unsubstituted amine groups, such as the products sold, for example under the name GP 4 SILICONE FLUID and GP 7100 by the company Genesee, or the products sold, for instance, under the names Q2 8220 and DOW CORNING 929 or 939 by the company Dow Corning. The substituted amine groups include, for example, C₁-C₄ aminoalkyl groups;

thiol groups such as the products sold, for instance, under the names GP 72 A and GP 71 from Genesee;

alkoxylated groups such as the product sold, for example, under the name SILICONE COPOLYMER F-755 by SWS Silicones and ABIL WAX 2428, 2434 and 2440 by the company Goldschmidt;

hydroxylated groups such as the polyorganosiloxanes comprising a hydroxyalkyl functional group, such as those described in French Patent Application No. FR-A-85/16334;

acyloxyalkyl groups such as the polyorganosiloxanes described in U.S. Pat. No. 4,957,732;

anionic groups of the carboxylic type, for example, the products described in European Patent Application No. 0 186 507 from the company Chisso Corporation, or of alkylcarboxylic type, such as those in the product X-22-3701E from the company Shin-Etsu;

2-hydroxyalkyl sulphonate; 2-hydroxyalkyl thiosulphate such as the products sold, for instance, by the company Goldschmidt under the names ABIL S201 and ABIL S255;

hydroxyacylamino groups, such as the polyorganosiloxanes described in European Patent Application No. 0 342 834. Mention may, for example, be made of the product Q2-8413 from the company Dow Corning.

Also useful herein are silicones comprising a polysiloxane portion and a portion comprising a nonsilicone organic chain, wherein one of the two portions constitutes the main chain of the polymer, and the other is grafted onto the main chain. These polymers are described, for example, in European Patent Application Nos. 0 412 704, 0 412 707, 0 640 105, and 0 582 152, and International Application Publication Nos. WO 95/00578, and WO 93/23009 and U.S. Pat. Nos. 4,693,935, 4,728,571 and 4,972,037. In at least one embodiment of the present disclosure, these polymers are anionic or nonionic.

According to the present disclosure, all of the silicones disclosed herein, may be used as such, and can also be used in the form of emulsions, nanoemulsions or microemulsions.

According to at least one embodiment of the present disclosure, the polyorganosiloxanes that are suitable for use may include: nonvolatile silicones chosen from polyalkylsiloxanes comprising trimethylsilyl end groups, such as oils having a viscosity ranging from 0.2 m²/s to 2.5 m²/s at 25° C., such as the oils of the DC200 series from Dow Corning, for example oils with a viscosity of 60,000 cSt, of the SILBIONE 70047 and 47 series and further for example, the oil 70,047 V 500,000, sold by the company Rhodia Chimie, polyalkylsiloxanes comprising dimethylsilanol end groups, such as dimethiconols, or polyalkylarylsiloxanes such as the oil SILBIONE 70641 V 200 sold by the company Rhodia Chimie;

the organopolysiloxane resin sold, for example, under the name DOW CORNING 593;

polysiloxanes comprising amine groups, such as amodimethicones or trimethylsilylamodimethicones.

In at least one embodiment, the cationic proteins or cationic protein hydrolysates are, for example, chemically modified polypeptides bearing quaternary ammonium groups at the end of the chain or grafted thereto. Their molecular mass can range, for example, from 1,500 to 10,000, such as ranging from 2,000 to 5,000. Examples of suitable cationic proteins or cationic protein hydrolysates include, but are not limited to:

collagen hydrolysates bearing triethylammonium groups; such as the products sold, for instance, under the name QUAT-PRO E by the company Maybrook and referred to in the CTFA dictionary as “Triethonium Hydrolyzed Collagen Ethosulfate”;

collagen hydrolysates bearing trimethylammonium and trimethylstearylammonium chloride groups, sold, for example, under the name QUAT-PRO S by the company Maybrook and referred to in the CTFA dictionary as “Steartrimonium Hydrolyzed Collagen”;

animal protein hydrolysates bearing trimethylbenzylammonium groups such as the products sold, for instance, under the name CROTEIN BTA by the company Croda and referred to in the CTFA dictionary as “Benzyltrimonium hydrolyzed animal protein”; and

protein hydrolysates bearing, on the polypeptide chain, quaternary ammonium groups comprising at least one C₁-C₁₈ alkyl radical.

Examples of protein hydrolysate sold by the company, Croda include, but are not limited to:

CROQUAT L, wherein the quaternary ammonium groups comprise a C₁₂ alkyl group;

CROQUAT M, wherein the quaternary ammonium groups comprise C₁₀-C₁₈ alkyl groups;

CROQUAT S, wherein the quaternary ammonium groups comprise a C₁₈ alkyl group;

CROTEIN Q, wherein the quaternary ammonium groups comprise at least one C₁-C₁₈ alkyl group.

Also useful herein are quaternized proteins or hydrolysates chosen from those of formula (XIV):

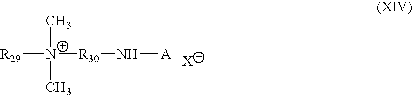

wherein

X⁻ is chosen from anions of an organic acid and anions of a mineral acid;

A is a protein residue derived from hydrolysates of collagen protein;

R₂₉ is a lipophilic group comprising up to 30 carbon atoms; and

R₃₀ is a C₁-C₆ alkylene group. Non-limiting mention may be made, for example, of the products sold by the company Inolex under the name LEXEIN QX 3000, referred to in the CTFA dictionary as “Cocotrimonium Collagen Hydrolysate”.

Non-limiting mention may also be made of quaternized plant proteins such as wheat, corn or soybean proteins: as quaternized wheat proteins, non-limiting mention may be made of those sold, for example, by the company Croda under the names HYDROTRITICUM WQ or QM, referred to in the CTFA dictionary as “Cocodimonium Hydrolysed Wheat Protein”, HYDROTRITICUM QL, referred to in the CTFA dictionary as “Laurdimonium Hydrolysed Wheat Protein” or HYDROTRITICUM QS, referred to in the CTFA dictionary as “Steardimonium Hydrolysed Wheat Protein”.

According to at least one embodiment of the present disclosure, the compounds of ceramide type are, for example, natural or synthetic ceramides, glycoceramides, pseudoceramides and neoceramides.

Non-limiting examples of compounds of ceramide type are described, for example, in German Patent Application Nos. 4 424 530, 4 424 533, 4 402 929, and 4 420 736, and International Patent Application Publication Nos. WO 95/23807, WO 95/16665, WO 94/07844, WO 94/24097, WO 94/10131 and WO 94/07844, and European Patent Application Nos. 0 646 572, and 0 227 994, and French Patent Application No. 2 673 179, the teachings of which are included herein by way of reference.

Compounds of ceramide type used in at least one embodiment of the present disclosure may include:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol, such as N-stearoylphytosphingosine,
2-N-palmitoylaminohexadecane-1,3-diol,
bis(N-hydroxyethyl-N-cetyl)malonamide,
N-(2-hydroxyethyl)-N-(3-cetyloxy-2-hydroxypropyl)cetylamide,
N-docosanoyl-N-methyl-D-glucamine,
   and mixtures thereof.

Cationic surfactants that may be useful herein include: optionally polyoxy-alkylenated primary, secondary and tertiary fatty amine salts, quaternary ammonium salts, imidazoline derivatives, and amine oxides of cationic nature.

Non-limiting examples of quaternary ammonium salts include:

those of formula (XV):

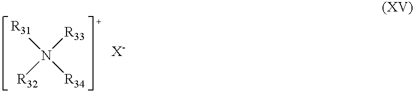

wherein:

R₃₁ to R₃₄, which may be identical or different, are chosen from C₁-C₃₀ linear and branched aliphatic radicals and aromatic radicals, such as aryl or alkylaryl. The aliphatic radicals may comprise heteroatoms such as oxygen, nitrogen, sulfur and halogens. The aliphatic radicals are chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkylacetate and hydroxyalkyl radicals, comprising from 1 to 30 carbon atoms;

X⁻ is an anion chosen from halides, phosphates, acetates, lactates, (C₂-C₆)alkyl sulfates, alkyl sulfonates and alkylaryl sulfonates;

quaternary ammonium salts of imidazolinium, such as the salt of formula (XVI):

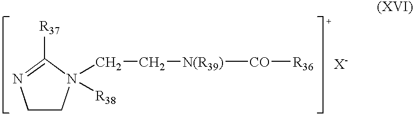

wherein:

R₃₆ is chosen from C₈-C₃₀ alkenyl radicals and C₈-C₃₀ alkyl radicals, for example tallow fatty acid derivatives;

R₃₇ is chosen from a hydrogen atom, C₁-C₄ alkyl radicals, C₈-C₃₀ alkenyl radicals and C₈-C₃₀ alkyl radicals;

R₃₈ is a C₁-C₄ alkyl radical;

R₃₉ is chosen from a hydrogen atom and C₁-C₄ alkyl radicals;

X⁻ is an anion chosen from halides, phosphates, acetates, lactates, alkyl sulfates, alkyl sulfonates and alkylaryl sulfonates.

R₃₆ and R₃₇, in at least one embodiment, are chosen from a mixture of C₁₂-C₂₁ alkenyl radicals and C₁₂-C₂₁alkyl radicals, for example tallow fatty acid derivatives; R₃₈ is a methyl radical and R₃₉ is a hydrogen atom. Such a product is sold, for example, under the name “REWOQUAT W 75” by the company Degussa;

diquaternary ammonium salts of formula (XVII):

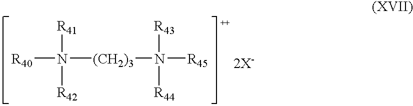

wherein:

R₄₀ is a C₁₆-C₃₀ aliphatic radical;

R₄₁, R₄₂, R₄₃, R₄₄ and R₄₅, which may be identical or different, are chosen from a hydrogen atom and C₁-C₄ alkyl radicals; and

X⁻ is an anion chosen from halides, acetates, phosphates, nitrates and methyl sulfates. A non-limiting example of diquaternary ammonium salts includes propane tallow diammonium dichloride.

According to at least one embodiment of the present disclosure, the quaternary ammonium salts comprising at least one ester function that may be used, include those of formula (XVIII):

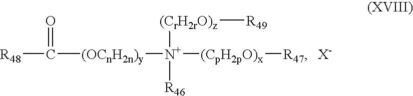

wherein:

R₄₆ is chosen from C₁-C₆ alkyl radicals, C₁-C₆ hydroxyalkyl and C₁-C₆ dihydroxyalkyl radicals;

R₄₇ is chosen from:
a radical

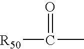

linear and branched, saturated and unsaturated C₁-C₂₂ hydrocarbon-based radicals;

R₅₁ is a hydrogen atom,

R₄₉ is chosen from:
a radical

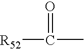

linear and branched, saturated and unsaturated C₁-C₆ hydrocarbon-based radicals;

R₅₃ is a hydrogen atom,

R₄₈, R₅₀ and R₅₂, which may be identical or different, are chosen from linear and branched, saturated and unsaturated C₇-C₂₁ hydrocarbon-based radicals;

n, p and r, which may be identical or different, are integers ranging from 2 to 6;

y is an integer ranging from 1 to 10;

x and z, which may be identical or different, are integers ranging from 0 to 10;

X⁻ is a simple or complex organic or inorganic anion; and

with the proviso that the sum x+y+z ranges from 1 to 15, that when x is 0, then R₄₇ is R₅₁ and that when z is 0, then R₄₉ is R₅₃.

The R₄₆ alkyl radicals may be linear or branched, and in at least one embodiment, linear.

R₄₆ may be a methyl, ethyl, hydroxyethyl or dihydroxypropyl radical and in at least one embodiment, a methyl or ethyl radical.

In at least one embodiment, the sum x+y+z is, for example, from 1 to 10.

When R₄₇ is a hydrocarbon-based radical R₅₁, it may be long and comprise from 12 to 22 carbon atoms, or short and comprise from 1 to 3 carbon atoms.

When R₄₉ is a hydrocarbon-based radical R₅₃, it comprises 1 to 3 carbon atoms.

R₄₈, R₅₀ and R₅₂, which may be identical or different, are chosen from linear and branched, saturated and unsaturated C₁₁-C₂₁ hydrocarbon-based radicals.

In another embodiment, R₄₈, R₅₀ and R₅₂ may be chosen from linear and branched, saturated and unsaturated, C₁₁-C₂₁ alkyl radicals and C₁₁-C₂₁ alkenyl radicals.

x and z, which may be identical or different, are in at least one embodiment, 0 or 1.

In at least one embodiment, y is 1.

In another embodiment, n, p and r, which may be identical or different, are 2 or 3.

The anion is, in at least one embodiment, a halide, for example, chloride, bromide and iodide, or an alkyl sulfate, for example, methyl sulfate. In another embodiment, methanesulfonate, phosphate, nitrate, tosylate, an anion derived from an organic acid, such as acetate or lactate, or any other anion that is compatible with the ammonium containing an ester function, may be used.

The anion X⁻ is, in at least one embodiment, chosen from chloride or methyl sulfate.

In at least one embodiment, the ammonium salts of formula (XVIII) may be used, wherein:

R₄₆ is a methyl or ethyl radical,

x and y are 1;

z is 0 or 1;

n, p and r are 2;

R₄₇ is chosen from:
a radical

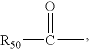

methyl, ethyl and C₁₄-C₂₂ hydrocarbon-based radicals,
and a hydrogen atom;

R₄₉ is chosen from:
a radical

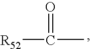

and a hydrogen atom;

R₄₈, R₅₀ and R₅₂, which may be identical or different, are chosen from linear and branched, saturated and unsaturated C₁₃-C₁₇ hydrocarbon-based radicals. In at least one embodiment, R₄₈, R₅₀ and R₅₂ and chosen from linear and branched, saturated and unsaturated C₁₃-C₁₇ alkyl and alkenyl radicals.

According to at least one embodiment, the hydrocarbon-based radicals are linear.

According to the present disclosure, the compounds of formula (XVI) may be chosen from, for example, diacyloxyethyldimethylammonium, diacyloxyethylhydroxyethylmethylammonium, monoacyl-oxyethyldihydroxyethylmethylammonium, triacyloxyethylmethylammonium and monoacyloxyethylhydroxyethyldimethylammonium salts, for instance, chloride and methyl sulfate, and mixtures thereof. The acyl radicals may comprise 14 to 18 carbon atoms and may be derived, for example, from a plant oil such as palm oil or sunflower oil. When the compound comprises several acyl radicals, these radicals may be identical or different.

These products are obtained, for example, by direct esterification of triethanolamine, triisopropanolamine, an alkyldiethanolamine or an alkyldiisopropanolamine, which may be oxyalkylenated, with fatty acids or with fatty acid mixtures of plant or animal origin, or by transesterification of the methyl esters thereof. This esterification is followed by a quaternization using an alkylating agent such as an alkyl halide, for example, a methyl and ethyl halide, a dialkyl sulfate, for instance, dimethyl and diethyl sulfate, methyl methanesulfonate, methyl para-toluene-sulfonate, glycol chlorohydrin and glycerol chlorohydrin.

Such compounds are sold, for example, under the names DEHYQUART by the company Cognis, STEPANQUAT by the company Stepan, NOXAMIUM by the company CECA or REWOQUAT WE 18 by the company Degussa.

The ammonium salts, which may comprise at least one ester function, and may be used in the context of the present disclosure are further described in U.S. Pat. Nos. 4,874,554 and 4,137,180.

According to the present disclosure, non-limiting examples of quaternary ammonium salts of formula (XV) that may be used herein are chosen from tetraalkylammonium chlorides such as dialkyldimethylammonium chlorides and alkyltrimethylammonium chlorides, wherein the alkyl radical comprises from 12 to 22 carbon atoms, for example, behenyltrimethylammonium chloride, distearyldimethylammonium chloride, cetyltrimethylammonium chloride, benzyldimethylstearylammonium chloride, and stearamidopropyldimethyl(myristyl acetate)ammonium chloride sold, for example, under the name CERAPHYL 70 by the company Van Dyk.

The saturated fatty acids, in at least one embodiment, are chosen from myristic acid, palmitic acid, stearic acid, behenic acid and isostearic acid.

In a further embodiment, the fatty acid esters other than those of the present disclosure, are chosen from carboxylic acid esters, such as mono-, di-, tri- and tetracarboxylic esters.

The monocarboxylic acid esters are different from those of the present disclosure and may, in at least one embodiment, be chosen from monoesters of linear and branched, saturated and unsaturated C₁-C₂₆ aliphatic acids, linear and branched, saturated and unsaturated C₁-C₂₆ aliphatic alcohols, the total carbon number of these esters is greater than or equal to 10.

Suitable monoesters include, by way of non-limiting example: dihydroabietyl behenate, octyldodecyl behenate, isocetyl behenate, cetyl lactate, C₁₂-C₁₅ alkyl lactate, isostearyl lactate, lauryl lactate, linoleyl lactate, oleyl lactate, (iso)stearyl octanoate, isocetyl octanoate, octyl octanoate, cetyl octanoate, decyl oleate, isocetyl isostearate, isocetyl laurate, isocetyl stearate, isodecyl octanoate, isodecyl oleate, isononyl isononanoate, isostearyl palmitate, methylacetyl ricinoleate, myristyl stearate, octyl isononanoate, 2-ethylhexyl isononate, octyl palmitate, octyl pelargonate, octyl stearate, octyldodecyl erucate, oleyl erucate, ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isostearyl neopentanoate, and isodecyl neopentanoate.

Other esters useful herein include, by way of non-limiting example, C₄-C₂₂ di- or tricarboxylic acid esters of C₁-C₂₂ alcohols and mono-, di- or tricarboxylic acid esters of C₂-C₂₆ di-, tri-, tetra- or pentahydroxy alcohols.

In at least one embodiment, non-limiting mention may also be made of: diethyl sebacate, diisopropyl sebacate, diisopropyl adipate, di-n-propyl adipate, dioctyl adipate, diisostearyl adipate, dioctyl maleate, glyceryl undecylenate, octyldodecylstearoyl stearate, pentaerythrityl monoricinoleate, pentaerythrityl tetraisononanoate, pentaerythrityl tetrapelargonate, pentaerythrityl tetraisostearate, pentaerythrityl tetraoctanoate, propylene glycol dicaprylate dicaprate, tridecyl erucate, triisopropyl citrate, triisostearyl citrate, glyceryl trilactate, glyceryl trioctanoate, trioctyldodecyl citrate, and trioleyl citrate.

Among the esters mentioned above, in at least one embodiment, the following non-limiting examples may be used herein: ethyl and isopropyl palmitates, 2-ethylhexyl palmitate, 2-octyldecyl palmitate, alkyl myristates such as isopropyl, butyl, cetyl or 2-octyldodecyl myristate, hexyl stearate, butyl stearate, isobutyl stearate; dioctyl malate, hexyl laurate, 2-hexyldecyl laurate, isononyl isononanoate, cetyl octanoate, isostearyl neopentanoate, and isodecyl neopentanoate.

The fluoro oils are, for example, the perfluoropolyethers described in European Patent Application No. 0 486 135 and the fluorohydrocarbon compounds described in International Patent Application Publication No. WO 93/11103, both of which are incorporated herein by way of reference.

As used herein, “fluorohydrocarbon compounds” are compounds with a chemical structure comprising a carbon skeleton wherein certain hydrogen atoms have been replaced with fluorine atoms.

The fluoro oils can also be fluorocarbons such as fluoroamines, for example, perfluorotributylamine, and fluorohydrocarbons, for example, perfluorodecahydronaphthalene, fluoro esters and fluoro ethers.

The perfluoropolyethers are sold, for example, under the trade names FOMBLIN by the company Montefluos and KRYTOX by the company Du Pont.

Among the fluorohydrocarbon compounds that may be mentioned are the esters of fluorine-containing fatty acid esters such as the product sold, for example, under the name NOFABLE FO by the company Nippon Oil.

The composition of the present disclosure may also comprise at least one mixture of conditioning agents.

Accordingly, the at least one conditioning agent can be present in an amount ranging from 0.001% to 20% by weight, for example, from 0.01% to 10% by weight, and further for example, from 0.1% to 3% by weight, relative to the total weight of the final composition.

The compositions for wash-protecting the color of keratin fibers according to the present disclosure, may be in the form of aqueous or aqueous-alcoholic haircare lotions, oils, gels, milks, creams, emulsions, and in the form of a mousse.

According to at least one embodiment, the compositions for wash-protecting the color of keratin fibers, may be in the form of shampoos, hair conditioners and pre-shampoo compositions.

The compositions for wash-protecting the color of keratin fibers, according to the present disclosure, may be packaged in various forms that include: vaporizers, pump-dispenser bottles and aerosol containers in order to apply the composition in vaporized form and in the form of a mousse. Such packaging forms are indicated, for example, when it is desired to obtain a spray, a lacquer or a mousse for treating the hair.

In at least one embodiment of the present disclosure, the pH of the composition for wash-protecting keratin fibers can range from 1 to 11, for example, from 2 to 6, and can be adjusted to the desired value with acidifying or basifying agents that are known in the prior art for compositions applied to keratin fibers.

Non-limiting examples of basifying agents that may be included in the present disclosure are aqueous ammonia, alkali metal carbonates, alkanolamines such as monoethanolamine, diethanolamine and triethanolamine and also derivatives thereof, oxyethylenated and/or oxypropylenated hydroxylamines and ethylenediamines, sodium hydroxide, potassium hydroxide and compounds having the formula below:

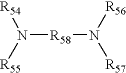

wherein:

R₅₈ is a propylene residue optionally substituted with a hydroxyl group or a C₁-C₄ alkyl radical;

R₅₄, R₅₅, R₅₆ and R₅₇, which may be identical or different, are chosen from a hydrogen atom, C₁-C₄ alkyl radicals and C₁-C₄ hydroxyalkyl radicals.

Non-limiting examples of acidifying agents that may be useful herein include, but are not limited to inorganic or organic acids such as hydrochloric acid, orthophosphoric acid, carboxylic acids such as tartaric acid, citric acid, lactic acid, and sulfonic acids.

Also disclosed herein is a process for wash-protecting the color of artificially dyed keratin fibers, wherein the process comprises applying to the fibers, before or after dyeing, at least one composition comprising, in a cosmetically acceptable medium, at least one liquid aromatic carboxylic acid ester as defined above.

In at least one embodiment, the composition comprising the at least one liquid aromatic carboxylic acid ester will be applied to said fibers after the dyeing step, which may be preceded or followed by a rinsing step and/or washing step with shampoo.

In another embodiment, the dyeing will be oxidation dyeing.

The process according to the present disclosure may comprise an additional step of total or partial drying of the keratin fibres with a hairdryer. According to another embodiment of the present disclosure, the process for wash-protecting the color of keratin fibers may comprise a step of heating the composition comprising the at least one multi-carbosite, multi-group coupling agent at a temperature less than or equal to 120° C., for example, 70° C., which will then be applied directly to the keratin fibers.

According to another embodiment, the process for wash-protecting the color of keratin fibers may comprise a step of heating the keratin fibers after application of the composition comprising the at least one multi-carbosite, multi-group coupling agent.

The heating of the keratin fibers may, for example, be carried out with an iron or a liquid water/steam mixture or alternatively, by means of a heating hood.

The heating iron that may be useful in the context of the present disclosure is a heating iron conventionally used in the haircare field, for example, a crimping iron or a smoothing iron. Other non-limiting examples of irons that may be useful herein are flat or round irons described, for instance, in U.S. Pat. Nos. 4,103,145, 4,308,878, 5,983,903, 5,957,140 and 5,494,058. The iron may be applied by successive separate touches for a few seconds, or by gradually moving or sliding it along the locks, or applying the iron to the keratin fibers after pausing. The pause will range, for example, from 30 seconds to 60 minutes, such as from 1 to 30 minutes. In at least one embodiment, the temperature ranges from 60° C. to 120° C.

The liquid water/steam mixture which may be used in accordance with the present disclosure may have a temperature of at least 35° C.

The liquid water/steam mixture may constitute a mist, and may comprise at least one other gas such as oxygen and nitrogen, and mixtures of gases such as air, and other vaporizable compounds.

In at least one embodiment, the temperature of the liquid water/steam mixture is greater than or equal to 40° C., for example, the temperature ranges from 40° C. to 75° C.

In another embodiment, the liquid water/steam mixture is brought into contact with the fiber for a period of time ranging from 1 second to 1 hour, for example from 5 minutes to 15 minutes. The application of the mixture can be repeated several times on the same fiber, each operation being performed for a period of time as indicated above. In another embodiment, the composition comprising the at least one multi-carbosite, multi-group coupling agent is first applied to the hair, and these locks are thus impregnated and then subjected to the action of the liquid water/steam mixture according to the conditions mentioned above, and the locks are thus treated and then cooled, for example, by sending over or through them a stream of cold air or of air at ambient temperature.

The liquid water/steam mixture, used in accordance with the present disclosure, can be produced using any apparatus known in the art and intended for this purpose. In at least one embodiment of the present disclosure, an apparatus comprising at least one steam generator directly connected to a hood that diffuses the liquid water/steam mixture onto the keratin fibers, such as human hair, may be used. A non-limiting example of this type of apparatus is that sold under the name ®MICROMIST by the company Takara Belmont.

According to at least one embodiment of the present disclosure, the dyeing process comprises applying to human keratin fibers, for example, hair, a direct or oxidation dye composition (A) for a period of time sufficient to develop the color, and following or preceding this application with the application of a composition (B) comprising, in a cosmetically acceptable medium, at least one multi-carbosite, multi-group coupling agent as defined above.

In at least one embodiment, the dyeing process is a one-step dyeing process comprising applying to keratin fibers, for example, human keratin fibers, such as the hair, a direct or oxidation dye composition (A′) for a period of time sufficient to develop the color, this composition (A′) comprising, in a cosmetically acceptable medium, at least one multi-carbosite, multigroup coupling agent as defined above.

In another embodiment, the dyeing is oxidation dyeing.

The application of the composition (A) or (A′) may be followed by rinsing and/or drying of the keratin fibers.

According to at least one embodiment, the application of the composition (B) may be followed by rinsing and/or drying of the keratin fibers. The composition (B) may be preheated under the conditions defined above, or the application of the composition (B) may be followed by heating of the keratin fibers under the same conditions defined above.

According to at least one embodiment, the composition (B) will be applied after the application of the direct or oxidation dye composition (A). The composition (B) comprising the at least one multi-carbosite, multi-group coupling agent, according to the present disclosure, may be applied immediately after dyeing or after a delay. As used herein, “after a delay” is intended to mean an application that takes place for a few hours or one or more days, for example, from 1 to 15 days, after dyeing. In at least one embodiment, the composition (B) will be applied immediately after dyeing the keratin fibers; the applications of the composition may be repeated between two colorings.

In the case of lightening direct dyeing operations, the dye compositions (A) or (A′) result from the mixing, at the time of use, of a dye composition (A₁) comprising at least one direct dye and of a composition (A₂) comprising at least one oxidizing agent.

In the case of oxidation dyeing, the dye compositions (A) or (A′) result from the mixing, at the time of use, of a dye composition (A₃) comprising at least one oxidation base and, optionally, at least one coupler and/or at least one direct dye, and of a composition (A₄) comprising at least one oxidizing agent.

Suitable direct dyes that may be used herein, are compounds that absorb light radiation in the visible range (400-750 nm), and may be nonionic, anionic or cationic in nature.

Non-limiting examples of the at least one direct dye include nitrobenzene dyes and azo, anthraquinone, naphthoquinone, benzoquinone, phenothiazine, indigoid, xanthene, phenanthridine, phthalocyanin and triaylmethane-based dyes, and mixtures thereof.

Useful nitrobenzene dyes that may be mentioned include, but are not limited to: 1-hydroxy-3-nitro-4-N-(γ-hydroxypropyl)aminobenzene, N-(β-hydroxyethyl)-amino-3-nitro-4-aminobenzene, 1-amino-3-methyl-4-N-(β-hydroxyethyl)amino-6-nitrobenzene, 1-hydroxy-3-nitro-4-N-(β-hydroxyethyl)aminobenzene, 1,4-diamino-2-nitrobenzene, 1-amino-2-nitro-4-methylaminobenzene, N-(β-hydroxyethyl)-2-nitro-para-phenylenediamine, 1-amino-2-nitro-4-(β-hydroxyethyl)amino-5-chlorobenzene, 2-nitro-4-aminodiphenylamine, 1-amino-3-nitro-6-hydroxybenzene, 1-(β-aminoethyl)-amino-2-nitro-4-(β-hydroxyethyloxy)benzene, 1-(β,γ-dihydroxypropyl)oxy-3-nitro-4-(β-hydroxyethyl)aminobenzene, 1-hydroxy-3-nitro-4-aminobenzene, 1-hydroxy-2-amino-4,6-dinitrobenzene, 1-methoxy-3-nitro-4-(β-hydroxyethyl)aminobenzene, 2-nitro-4′-hydroxydiphenylamine, 1-amino-2-nitro-4-hydroxy-5-methylbenzene, and mixtures thereof.

The nitrobenzene direct dyes may also be chosen from yellow dyes and green dyes, for instance, 1-β-hydroxyethyloxy-3-methylamino-4-nitrobenzene, 1-methylamino-2-nitro-5-(β,γ-dihydroxypropyl)oxybenzene, 1-(β-hydroxyethyl)amino-2-methoxy-4-nitrobenzene, 1-(β-aminoethyl)amino-2-nitro-5-methoxybenzene, 1,3-bis(β-hydroxyethyl)amino-4-nitro-6-chlorobenzene, 1-amino-2-nitro-6-methylbenzene, 1-(β-hydroxyethyl)amino-2-hydroxy-4-nitrobenzene, N-(β-hydroxyethyl)-2-nitro-4-trifluoromethylaniline, 4-(β-hydroxyethyl)amino-3-nitrobenzenesulphonic acid, 4-ethylamino-3-nitrobenzoic acid, 4-(β-hydroxyethyl)amino-3-nitrochlorobenzene, 4-(β-hydroxyethyl)amino-3-nitromethylbenzene, 4-(β,γ-dihydroxypropyl)amino-3-nitrotrifluoromethylbenzene, 1-(β-ureidoethyl)amino-4-nitrobenzene, 1,3-diamino-4-nitrobenzene, 1-hydroxy-2-amino-5-nitrobenzene, 1-amino-2-[tris(hydroxymethyl)-methyl]amino-5-nitrobenzene, 1-(β-hydroxyethyl)amino-2-nitrobenzene and 4-(β-hydroxyethyl)amino-3-nitrobenzamide.

Blue and violet nitrobenzene dyes that may be useful to the present disclosure include: 1-(β-hydroxyethyl)amino-4-N,N-bis(β-hydroxyethyl)amino-2-nitrobenzene, 1-(γ-hydroxypropyl)amino-4-N,N-bis(β-hydroxyethyl)amino-2-nitrobenzene, 1-(β-hydroxyethyl)amino-4-(N-methyl,N-β-hydroxyethyl)amino-2-nitrobenzene, 1-(β-hydroxyethyl)amino-4-(N-ethyl,N-β-hydroxyethyl)amino-2-nitrobenzene, 1-(β,γ-dihydroxypropyl)amino-4-(N-ethyl, N-β-hydroxyethyl)amino-2-nitrobenzene, and 2-nitro-para-phenylenediamines of the following formula:

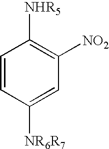

wherein:

R₆ is chosen from C₁-C₄ alkyl radicals, β-hydroxyethyl radicals, β-hydroxypropyl radical, and γ-hydroxypropyl radicals;

R₅ and R₇, which may be identical or different, are chosen from β-hydroxyethyl radicals, β-hydroxypropyl radicals, γ-hydroxypropyl radicals and β,γ-dihydroxypropyl radicals, wherein at least one of the radicals R₆, R₇ or R₅, is a γ-hydroxypropyl radical and R₆ and R₇ are not simultaneously β-hydroxyethyl radicals when R₆ is a γ-hydroxypropyl radical, such as those described in French Patent No. 2 692 572.

It is understood that azo dyes are compounds comprising in their structure at least one —N═N— sequence not included in a ring; methine dyes are compounds comprising in their structure at least one —C═C— sequence not included in a ring; and azomethine dyes are compounds comprising in their structure at least one —C═N— sequence not included in a ring.

In at least one embodiment, the triarylmethane-based dyes comprise in their structure at least one compound of formula:

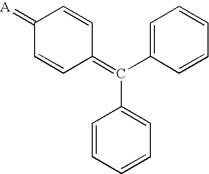

wherein:

A is chosen from an oxygen atom and a nitrogen atom.

In another embodiment, the xanthene dyes comprise in their structure at least one compound of formula:

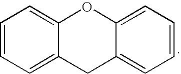

In yet another embodiment, the phenanthridine dyes comprise in their structure at least one compound of formula:

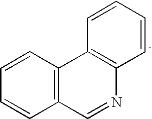

In another embodiment, the phthalocyanine dyes comprise in their structure at least one compound of formula:

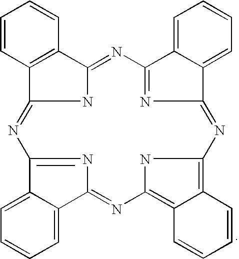

In yet another embodiment, the phenothiazine dyes comprise in their structure at least one compound of formula:

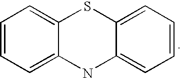

The direct dyes may also be chosen from basic dyes such as those listed in the Color Index, 3rd edition, for example, those under the names Basic Brown 16, Basic Brown 17, Basic Yellow 57, Basic Red 76, Basic Violet 10, Basic Blue 26 and Basic Blue 99; from acidic direct dyes listed in the Color Index, 3rd edition, under the names Acid Orange 7, Acid Orange 24, Acid Yellow 36, Acid Red 33, Acid Red 184, Acid Black 2, Acid Violet 43, and Acid Blue 62, and cationic direct dyes such as those described in International Patent Application Publication Nos. WO 95/01772, and WO 95/15144, and European Patent Application No. 0 714 954, the contents of which are incorporated herein in their entireties, for example, Basic Red 51, Basic Orange 31 and Basic Yellow 87.

According to at least one embodiment, the at least one direct dye that can be used in the processes as disclosed herein or that have been used for the artificial dyeing of the hair before use of the at least one multi-carbosite, multi-group coupling agent according to the present disclosure, are ionic in nature, for example, cationic.

When they are present, the at least one direct dye is present in the composition in an amount ranging from 0.0005% to 12% by weight, for example, from 0.005% to 6% by weight, relative to the total weight of the dye composition.

The oxidation bases may be chosen from oxidation bases conventionally used in oxidation dyeing, for example, para-phenylenediamines, bisphenylalkylenediamines, paraminophenols, ortho-aminophenols and heterocyclic bases.

Non-limiting examples of para-phenylenediamines include, but are not limited to para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, N,N-diethyl-4-amino-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine and N-(β-methoxyethyl)-para-phenylenediamine, and acid addition salts thereof.

In at least one embodiment, the para-phenylenediamines may be chosen from: para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the acid addition salts thereof.

Non-limiting examples of the bis(phenyl)alkylenediamines include: N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-amino-phenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,5-dioxaoctane, and the acid addition salts thereof.

Suitable para-aminophenols may be chosen, by way of non-limiting example, from para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the acid addition salts thereof.

Suitable ortho-aminophenols include, but are not limited to 2-aminophenol, 2-amino-5-methylphenol, 2-amino-6-methylphenol and 5-acetamido-2-aminophenol, and the acid addition salts thereof.

Suitable heterocyclic bases include, by way of non-limiting example, pyridine derivatives, pyrimidine derivatives and pyrazole derivatives.

Non-limiting examples of pyridine derivatives include the compounds described in British Patent Nos. 1 026 978 and 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the acid addition salts thereof.

Suitable pyrimidine derivatives include, by way of non-limiting example, the compounds described in German Patent No. 2 359 399; Japanese Patent Nos. 88-169 571 and 05-163 124; European Patent No. 0 770 375 and International Patent Application Publication No. WO 96/15765, such as 2,4,5,6-tetraminopyrimidine, 4-hydroxy-2,5,6-triaminopyrimidine, 2-hydroxy-4,5,6-triaminopyrimidine, 2,4-dihydroxy-5,6-diaminopyrimidine and 2,5,6-triaminopyrimidine, and pyrazolopyrimidine derivatives such as those mentioned in French Patent Application No. 2 750 048 such as pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl) (2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine and 3-amino-5-methyl-7-imidazolylpropylaminopyrazolo[1,5-a]pyrimidine, and the acid addition salts thereof, and the tautomeric forms thereof, when a tautomeric equilibrium exists.

Suitable pyrazole derivatives may include, but are not limited to compounds described in German Patent Nos. 3 843 892, 195 43 988 and 4 133 957 and International Patent Application Publication Nos. WO 94/08969, and WO 94/08970, and French Application No. 2 733 749, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the acid addition salts thereof.

When present, these oxidation bases are present in an amount ranging from 0.0005% to 12% by weight, for example, from 0.005% to 6% by weight, relative to the total weight of the dye composition.

The oxidation dye compositions in accordance with the present disclosure may also comprise at least one coupler and/or at least one direct dye, to modify the shades and/or to enrich them with tints.

Non-limiting examples of the at least one coupler that can be used in the oxidation dye compositions according to the present disclosure include couplers conventionally used in oxidation dyeing, such as meta-phenylenediamines, meta-aminophenols, meta-diphenols, naphthols and heterocyclic couplers, for instance indole derivatives, indoline derivatives, pyridine derivatives, indazole derivatives, pyrazolo[1,5-b]-1,2,4-triazole derivatives, pyrazolo[3,2-c]-1,2,4-triazole derivatives, benzimidazole derivatives, benzothiazole derivatives, benzoxazole derivatives, 1,3-benzodioxole derivatives and pyrazolones, and the acid addition salts thereof.

According to at least one embodiment, the at least one coupler may be chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, sesamol, α-naphthol, 2-methyl-1-naphthol, 6-hydroxyindole, 4-hydroxyindole, 4-hydroxy-N-methylindole, 6-hydroxyindoline, 6-hydroxybenzomorpholine, 3,5-diamino-2,6-dimethoxypyridine, 1-N-(β-hydroxyethyl)-amino-3,4-methylenedioxybenzene, 2,6-bis(β-hydroxyethyleneamino)toluene, 2,6-dihydroxy-4-methylpyridine, 1H-3-methylpyrazol-5-one, 1-phenyl-3-methylpyrazol-5-one, and the acid addition salts thereof.

When present, the at least one coupler is present in an amount ranging from 0.0001% to 10% by weight, for example, from 0.005% to 5% by weight, relative to the total weight of the oxidation dye composition.

According to at least one embodiment, the oxidation dyes that may be used in the processes as disclosed herein, or that have been used for the artificial dyeing of the hair before use of the at least one multi-carbosite, multi-group coupling agent as disclosed herein, comprise at least one oxidation base of para-phenylenediamine type or of para-aminophenol type.

The dye composition in accordance with the present disclosure, may also comprise at least one adjuvant conventionally used in hair dye compositions, such as anionic, cationic, nonionic, amphoteric, zwitterionic surfactants and mixtures thereof; anionic, cationic, nonionic, amphoteric, zwitterionic polymers and mixtures thereof; inorganic and organic thickeners; antioxidants; penetrating agents; sequestering agents; fragrances; buffers; dispersants; conditioning agents such as silicones; film-forming agents; preservatives and opacifiers.

It is to be understood that a person skilled in the art will take care to select the at least one optional additional compound in such that the beneficial properties intrinsically associated with the composition in accordance with the present disclosure are not, or are not substantially, adversely affected by the envisaged addition(s).

The dye composition according to the present disclosure may be in various forms, such as in the form of liquids, creams and gels, or in any other form that is suitable for dyeing keratin fibers, for example, human hair.

The at least one oxidizing agent in the lightening direct dyeing operation (direct dyeing with an oxidizing agent) or in the oxidation dyeing operation may, by way of non-limiting example, be chosen from hydrogen peroxide, urea peroxide, alkali metal bromates or ferricyanides, and persalts such as perborates and persulfates. Redox enzymes such as laccases, peroxidases and 2-electron oxidoreductases (such as uricase) may also be used as the at least one oxidizing agent, where appropriate in the presence of their respective donor or cofactor thereof.

According to at least one embodiment of the present disclosure, the process may be used on hair that has been sensitized by hair treatments other than those mentioned in the present disclosure.

Another aspect of the present disclosure is a multicomponent dyeing kit comprising at least one first component comprising a direct dye composition (A) and at least one second component comprising a composition (B) comprising, in a cosmetically acceptable medium, at least one multi-carbosite, multi-group coupling agent according to the present disclosure.

Still another aspect of the present disclosure is a multicomponent dyeing kit comprising at least one first component comprising a composition (A₁) comprising at least one direct dye, at least one second component comprising a composition (A₂) comprising at least one oxidizing agent, and at least one third component comprising a composition (B) comprising, in a cosmetically acceptable medium, at least one multi-carbosite, multi-group coupling agent according to the present disclosure.

Disclosed herein is also a multicomponent dyeing kit comprising at least one first component comprising a composition (A₃) comprising at least one oxidation base and, optionally, at least one coupler and/or at least one direct dye, at least one second component comprising a composition (A₄) comprising at least one oxidizing agent, and a third component comprising at least one composition (B) comprising, in a cosmetically acceptable medium, at least one multi-carbosite, multi-group coupling agent according to the present disclosure.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

The examples that follow are intended to illustrate the present disclosure

Without, however, being limiting in nature.

### EXAMPLES

The compositions according to the present disclosure, that can be used for wash-protecting the color of keratin fibers, are exemplified by the following nonexhaustive list:

### Example 1

### Rinse-Out Lotions

The rinse-out lotions were prepared from the following ingredients in the following proportions given as % by weight.

| Ingredient | Lotion 1 | Lotion 2 | Lotion 3 | Lotion 4 | Lotion 5 |
|---|---|---|---|---|---|
| Ethanol | 10% | — | — | — | 30% |
| Active | 5% | — | — | — | — |
| agent 1 | | | | | |
| Active | — | 5% | — | — | — |
| agent 2 | | | | | |
| Active | — | — | 0.5 | — | — |
| agent 3 | | | | | |
| Active | — | — | — | 5% | — |
| agent 4 | | | | | |
| Active | — | — | — | — | 1% |
| agent 5 | | | | | |
| Preservative | qs | qs | qs | qs | qs |
| Fragrance | qs | qs | qs | qs | qs |
| Water | qs 100 | qs 100 | qs 100 | qs 100 | qs 100 |

### Active Agents

| | | |
|---|---|---|
| 1 | 1,3-Butadiene diepoxide, CAS = 1464-53-5, sold by Aldrich under thereference 202533 | 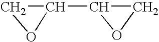 |
| 2 | Triethanolamine trisulfate asdescribed in the article “Synthesis oftriethanolamine trisulphate and itsapplication to cellulosic fabrics”.Lewis, D. M.; Zhao, X, F. Departmentof Colour and Polymer chemistry,University of Leeds, UK. ColorationTechnology (2004), 120(4) 172-179 | 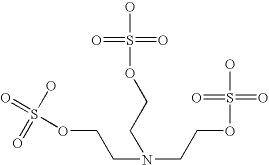 |
| 3 | Ethylenediaminetetraaceticdianhydride, CAS = 23911-25-3, soldby Aldrich under the reference 332046 | 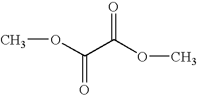 |
| 4 | Sodium sulphosuccinimidyl-4-(N-maleimidomethyl)cyclohexane-1-CAS = 92921-24-9, sold by Pierceunder the reference SULFO-SMCC | 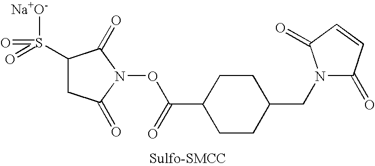 |
| 5 | Dimethyl oxalate | 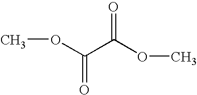 |

### Example 2

### Rinse-Out Compositions

The shampoos were prepared from the following ingredients in the following amounts given as % by weight of active material.

| | Ex. 2 |
|---|---|
| Sodium laurethsulfate⁽¹⁾ | 30 AM |
| Cocobetaine⁽²⁾ | 4 AM |
| Cocamide MIPA⁽³⁾ | 2 AM |
| PDMS (MW 250 000)⁽⁴⁾ | 2 AM |
| Active agent 1 | 5 AM |
| Sodium cetearyl sulfate⁽⁵⁾ | 0.8 AM |
| Polyquaternium-10⁽⁶⁾ | 0.4 AM |
| Carbomer⁽⁷⁾ | 0.2 AM |
| Propylene glycol | 0.1 AM |
| Preservative | qs |
| Fragrance | qs |
| Water qs | 100 |

A.M.: active material
⁽¹⁾TEXAPON N 702 sold by the company Cognis,
⁽²⁾DEHYTON AB 30 OR sold by the company Cognis,
⁽³⁾EMPILAN CIS sold by the company Huntsman,
⁽⁴⁾MIRASIL DM 500 000 sold by the company Rhodia,
⁽⁵⁾LANETTE E sold by the company Cognis,
⁽⁶⁾UCARE Polymer JR 400 sold by the company Amerchol,
⁽⁷⁾CARBOPOL 980 sold by the company Noveon.

### Example 3

### Leave-In Compositions

The glossing creams were prepared from the following ingredients in the following amounts given as % by weight of active material.

| | Ex. 3 |
|---|---|
| Cyclopentasiloxane⁽¹⁾ | 5 AM |
| Active agent 1 | 0.5 AM |
| Cyclopentasiloxane dimethicone copolyol⁽²⁾ | 0.5 AM |
| Propylene glycol | 2.5 AM |
| Preservative | qs |
| Fragrance | qs |
| Water qs | 100 |

A.M.: active material
⁽¹⁾MIRASOL CM 5 sold by the company Rhodia,
⁽²⁾DOW CORNING 5225C sold by the company Dow Corning,

### Example 4

### Application Protocol

Lotion 2 comprising active agent 2 was applied to a lock of permanent-waved hair dyed with the shade MAJIROUGE 6.66, using heat as activator.

The process was carried out as follows:

lotion 2 was applied at a rate of 2 grams per gram of hair

5 passes of a smoothing iron at a temperature set at 100° C. were performed

the locks were rinsed with water

the locks were then shampooed with DOP camomile shampoo.

A satisfactory color wash-fastness was obtained.

What is claimed is:

1. A process for wash-protecting the color of artificially dyed keratin fibers comprising applying to the fibers at least one composition comprising at least one multi-carbosite, multi-group coupling agent,
wherein the at least one multi-carbosite, multi-group coupling agent is an electrophilic hydrocarbon-based compound, with the proviso that it does not comprise an aldehyde group, a carboxylic acid group or salts thereof, a formol-generating group, or an ammonium group,
wherein the multi-carbosite, multi-group coupling agent comprises at least two identical electrophilic reactive groups,
the reactive groups not being carried by the same atom, if said atom is a carbon atom, and wherein the coupling agent has a molecular weight of less than 500 g/mol.

2. The process according to claim 1, wherein the reactive groups of the at least one multi-carbosite, multi-group coupling agent are chosen from:
epoxides,
(C₂-C₃₀)alkyl sulfates,
aziridines,
activated cyclopropanes,
vinyls and activated vinyls,
alkyl esters,
alkylamides,
anhydrides,
acyl (or haloformyl) halides,
acetals and hemiacetals,
ketones,
lactones, thiolactones,
isocyanates,
isothiocyanate,
imides,
imidates
thiosulfates,
oxazines and oxazolines,
oxaziniums and oxazoliniums,
C₆ to C₃₀ alkyl, aryl and aralkyl halides of formula RX wherein X is chosen from 1, Br and Cl,
halides of an unsaturated ring which is carbon-based, or a heterocycle,
sulfonyl halides (—SO₂X), wherein X is chosen from Cl and F,
(C₂-C₃₀)alkyl phosphonium,
phenols,
(C₁-C₃₀)alkyl nitrophenol carbonates,
(C₁-C₃₀)alkyl or phenyl tosylates,
alkyl chloroformates,
enol esters,
sulfonamides, and
ring opening reactants,

3. The process according to claim 2, wherein the reactive groups of the at least one multi-carbosite, multi-group coupling agent are chosen from: aziridines, epoxides, (C₂-C₃₀)alkyl sulfates, vinyls, activated vinyls, oxalates, ketones, alpha-hydroxy ketones, alpha-halo ketones, lactones, thiolactones, isocyanates, isothiocyanates, oxazines, oxazolines, halides of an unsaturated ring which is carbon-based, heterocycles, sulfonyl halides, and (C₂-C₃₀)alkyl phosphoniums.

4. The process according to claim 1, wherein the at least one multi-carbosite, multi-group coupling agent is chosen from 1,3-butadiene diepoxide, triethanolamine trisulfate, dimethyl oxalate, ethylenediaminetetraacetic dianhydride and sodium sulfosuccinimidyl-4-(N-maleimidomethyl)cyclohexane-1-carboxylate.

5. The process according to claim 1, wherein the at least one multi-carbosite, multi-group coupling agent is present in the composition in an amount ranging from 0.01% to 20% by weight, relative to the total weight of the composition.

6. The process according to claim 5, wherein the at least one multi-carbosite, multi-group coupling agent is present in the composition in an amount ranging from 0.1% to 10% by weight, relative to the total weight of the composition.

7. A process for wash-protecting the color of artificially dyed keratin fibers, comprising applying to the keratin fibers, before or after dyeing the fibers, at least one composition comprising at least one multi-carbosite, multi-group coupling agent,
wherein the at least one multi-carbosite, multi-group coupling agent is an electrophilic hydrocarbon-based compound, with the proviso that it does not comprise an aldehyde group, a carboxylic acid group or salts thereof, a formol-generating group, or an ammonium group,
wherein the multi-carbosite, multi-group coupling agent comprises at least two identical electrophilic reactive groups,
the reactive groups not being carried by the same atom, if said atom is a carbon atom, and wherein the coupling agent has a molecular weight of less than 500 g/mol.

8. The process according to claim 7, wherein the composition comprising at least one multi-carbosite, multi-group coupling agent is applied to the fibers after dyeing the keratin fibers.

9. The process according to claim 7, wherein the composition further comprises at least one agent for protecting against the effects of atmospheric agents.

10. The process according to claim 7, wherein the composition further comprises at least one conditioning agent.

11. A multistep process for dyeing keratin fibers, comprising:
applying to the fibers a direct or oxidation dye composition (A) for a period of time sufficient to develop the color; and
following or preceding the application of the dye composition (A) with the application of a composition (B) comprising, in a cosmetically acceptable medium, at least one multi-carbosite, multi-group coupling agent,
wherein the at least one multi-carbosite, multi-group coupling agent is an electrophilic hydrocarbon-based compound, with the proviso that it does not comprise an aldehyde group, a carboxylic acid group or salts thereof, a formol-generating group, or an ammonium group,
wherein the multi-carbosite, multi-group coupling agent comprises at least two identical electrophilic reactive groups,
the reactive groups not being carried by the same atom, if said atom is a carbon atom, and wherein the coupling agent has a molecular weight of less than 500 g/mol.

12. A one-step dyeing process comprising applying to the keratin fibers, a direct or oxidation dye composition (A′) for a period of time sufficient to develop the color, this composition (A′) comprising, in a cosmetically acceptable medium, at least one multi-carbosite, multi-group coupling agent,
wherein the at least one multi-carbosite, multi-group coupling agent is an electrophilic hydrocarbon-based compound, with the proviso that it does not comprise an aldehyde group, a carboxylic acid group or salts thereof, a formol-generating group, or an ammonium group,
wherein the multi-carbosite, multi-group coupling agent comprises at least two identical electrophilic reactive groups,
the reactive groups not being carried by the same atom, if said atom is a carbon atom, and wherein the coupling agent has a molecular weight of less than 500 g/mol.

13. The process according to claim 11, wherein the composition (B) is applied to the keratin fibers, either immediately or after a delay, after the direct or oxidation dye composition (A) is applied to the keratin fibers,
it being possible to apply said composition (B) between two colorings.

14. A multicomponent dyeing kit comprising at least one first component comprising a direct or oxidation dye composition (A) and at least one second component comprising a composition (B) comprising, in a cosmetically acceptable medium, at least one multi-carbosite, multi-group coupling agent,
wherein the at least one multi-carbosite, multi-group coupling agent is an electrophilic hydrocarbon-based compound, with the proviso that it does not comprise an aldehyde group, a carboxylic acid group or salts thereof, a formol-generating group, or an ammonium group,
wherein the multi-carbosite, multi-group coupling agent comprises at least two identical electrophilic reactive groups,
the reactive groups not being carried by the same atom, if said atom is a carbon atom, and wherein the coupling agent has a molecular weight of less than 500 g/mol.

15. A multicomponent dyeing kit comprising at least one first component comprising a composition (A₁) comprising at least one direct dye, at least one second component comprising a composition (A₂) comprising at least one oxidizing agent, and at least one third component comprising a composition (B) comprising, in a cosmetically acceptable medium, at least one multi-carbosite, multi-group coupling agent,
wherein the at least one multi-carbosite, multi-group coupling agent is an electrophilic hydrocarbon-based compound, with the proviso that it does not comprise an aldehyde group, a carboxylic acid group or salts thereof, a formol-generating group, or an ammonium group,
wherein the multi-carbosite, multi-group coupling agent comprises at least two identical electrophilic reactive groups,
the reactive groups not being carried by the same atom, if said atom is a carbon atom, and wherein the coupling agent has a molecular weight of less than 500 g/mol.

16. A multicomponent dyeing kit comprising at least one first component comprising a composition (A₃) comprising at least one oxidation base and, optionally, at least one coupler and/or at least one direct dye, at least one second component comprising a composition (A₄) comprising at least one oxidizing agent, and at least one third component comprising a composition (B) comprising, in a cosmetically acceptable medium, at least one multi-carbosite, multi-group coupling agent;
wherein the at least one multi-carbosite, multi-group coupling agent is an electrophilic hydrocarbon-based compound, with the proviso that it does not comprise an aldehyde group, a carboxylic acid group or salts thereof, a formol-generating group, or an ammonium group,
wherein the multi-carbosite, multi-group coupling agent comprises at least two identical electrophilic reactive groups,
the reactive groups not being carried by the same atom, if said atom is a carbon atom, and wherein the coupling agent has a molecular weight of less than 500 g/mol.
